(19) United States
(12) Patent Application Publication
(10) Pub. No.: US 2026/0165590 A1
(43) Pub. Date: Jun. 18, 2026

(54) METHOD AND APPARATUS OF NON-CONTACT BLOOD PRESSURE MEASUREMENT BASED ON RADAR TECHNOLOGY

(71) Applicant: Guangdong AI Touch Technology Co., Ltd., Foshan (CN)

(72) Inventors: Huan Sun, Foshan (CN); Zhen Hu, Foshan (CN); Sihan Chen, Foshan (CN); Yining Duan, Foshan (CN)

(21) Appl. No.: 19/008,697

(22) Filed: Jan. 3, 2025

(30) Foreign Application Priority Data
Dec. 12, 2024 (CN) 202411836644.4

(51) Int. Cl. A61B 5/021; A61B 5/00; A61B 5/02; A61B 5/026; A61B 5/05; A61B 5/11; G16H 10/60

(52) U.S. Cl. CPC A61B 5/021; A61B 5/02007; A61B 5/026; A61B 5/05; A61B 5/1118; A61B 5/1126; A61B 5/4809; A61B 5/7264; G16H 10/60

(57) ABSTRACT

The present disclosure relates to the technical field of blood pressure measurement. Disclosed is a method and an apparatus of non-contact blood pressure measurement based on radar technology. The method includes: performing a signal detection operation on a target body part of a user to be measured by means of a radar detector to acquire a signal parameter of an echo signal of the user to be measured; identifying, according to the signal parameter of the echo signal, biological signal information of the user to be measured; and performing a blood pressure prediction operation, according to the biological signal information, on the user to be measured to acquire a blood pressure parameter of the user to be measured.

### CROSS REFERENCE TO RELEATED APPLICATIONS

The present disclosure claims the priority of Chinese Patent Application No. 2024118366444 filed on Dec. 12, 2024 before CNIPA. All the above are hereby incorporated by reference in their entirety.

### TECHNICAL FIELD

The present disclosure relates to the technical field of blood pressure measurement and, particularly, to a method and an apparatus of non-contact blood pressure measurement based on radar technology.

### BACKGROUND

Blood pressure is an important indicator of cardiovascular health, and maintaining an appropriate blood pressure level is essential to ensure the normal operation of various physiological functions of the human body. Regular blood pressure measurement helps to detect abnormal blood pressure fluctuations early, so that timely and effective interventions can be taken.

At present, electronic sphygmomanometers, as the mainstream contact-type blood pressure measurement tools, are widely favored for their ease of operation and relatively high accuracy. However, since this blood pressure measurement method is required to pressurize the cuff, it may cause a certain degree of physical discomfort to the subject, and the device cannot be used normally for special populations, such as disabled people or burn patients, thus affecting the measurement efficiency and measurement accuracy of blood pressure. In view of this, it is particularly important to propose a non-contact blood pressure measurement method based on radar technology.

### SUMMARY

Provided in the present disclosure is a method and an apparatus of non-contact blood pressure measurement based on radar technology, so that blood pressure can be measured in a non-contact manner, which improves the flexibility of the blood pressure measurement of the subject, which in turn improves the efficiency and accuracy of the blood pressure measurement of the subject, and reduces the discomfort of the measurement of the subject, which in turn improves the device use experience of the subject.

In order to solve the aforementioned technical problems, disclosed as a first aspect in the present disclosure is a method of non-contact blood pressure measurement based on radar technology, and the method includes:
performing a signal detection operation on a target body part of a user to be measured by means of a preset radar detector to acquire a signal parameter of an echo signal of the user to be measured;
identifying, according to the signal parameter of the echo signal, biological signal information of the user to be measured; and
performing a blood pressure prediction operation, according to the biological signal information of the user to be measured, on the user to be measured to acquire a blood pressure parameter of the user to be measured, wherein the blood pressure parameter includes a systolic blood pressure parameter and/or a diastolic blood pressure parameter.

As an optional implementation, in the first aspect of the present disclosure, the signal parameter of the echo signal includes at least one of a waveform increasing rate, a waveform peak, a waveform decreasing rate, a waveform period, a waveform frequency, and a waveform amplitude of the echo signal; the biological signal information of the user to be measured includes at least one of vascular elasticity degree, vascular blood flow rate, vascular blood flow resistance, vascular blood flow volume, and time of change of vascular blood flow of the user to be measured.

the performing, according to the biological signal information of the user to be measured, a blood pressure prediction operation on the user to be measured to acquire a blood pressure parameter of the user to be measured includes:
identifying, according to the biological signal information of the user to be measured, cardiac feature information of the user to be measured; the cardiac feature information of the user to be measured including cardiac systolic feature information and/or cardiac diastolic feature information of the user to be measured; and
performing, according to the cardiac feature information of the user to be measured, a blood pressure prediction operation on the user to be measured to acquire a blood pressure parameter of the user to be measured.

As an optional implementation, in the first aspect of the present disclosure, after performing, according to the biological signal information of the user to be measured, a blood pressure prediction operation on the user to be measured to acquire a blood pressure parameter of the user to be measured, the method further includes:
acquiring first target information of the user to be measured, the first target information including basic information and/or health condition information of the user to be measured, the basic information of the user to be measured including at least one of height, weight, gender, and body type of the user to be measured;
identifying, according to the first target information of the user to be measured, a first blood pressure prediction affected condition corresponding to the first target information of the user to be measured, and identifying, according to the first blood pressure prediction affected condition corresponding to the first target information of the user to be measured, a first blood pressure prediction affected level corresponding to the first target information of the user to be measured; and
judging whether the first blood pressure prediction affected level is greater than or equal to a preset first affected level threshold, and if yes, correcting the blood pressure parameter of the user to be measured according to the first target information.

As an optional implementation, in the first aspect of the present disclosure, after performing, according to the biological signal information of the user to be measured, a blood pressure prediction operation on the user to be measured to acquire a blood pressure parameter of the user to be measured, the method further includes:
acquiring second target information of the user to be measured, the second target information including at least one of activity state, sleep state, and psychological state of the user to be measured, the activity state including activity intensity and/or activity duration, and the sleep state including sleep depth and/or sleep duration;
identifying, according to the second target information of the user to be measured, a second blood pressure prediction affected condition corresponding to the second target information of the user to be measured, and identifying, according to the second blood pressure prediction affected condition corresponding to the second target information of the user to be measured, a second blood pressure prediction affected level corresponding to the second target information of the user to be measured; and
judging whether the second blood pressure prediction affected level is greater than or equal to a preset second affected level threshold, and if yes, correcting the blood pressure parameter of the user to be measured according to the second target information.

As an optional implementation, in the first aspect of the present disclosure, the performing a signal detection operation on a target body part of a user to be measured by means of a preset radar detector to acquire a signal parameter of an echo signal of the user to be measured includes:
acquiring first detecting reference information of the user to be measured, the first detecting reference information of the user to be measured including health condition and/or current activity of the user to be measured, the current activity including at least one of current activity intensity, current activity posture, current activity amplitude, and duration of activity already performed;
identifying a detecting control parameter corresponding to the preset radar detector according to the first detecting reference information of the user to be measured, the detecting control parameter including at least one of detecting position, detecting distance, detecting wave, detecting duration, and signal receiving position; and
performing, according to the detecting control parameter corresponding to the radar detector, the signal detection operation on the target body part of the user to be measured to acquire the signal parameter of the echo signal of the user to be measured.

As an optional implementation, in the first aspect of the present disclosure, before identifying a detecting control parameter corresponding to the preset radar detector according to the first detecting reference information of the user to be measured, the method further includes:
acquiring second detecting reference information of the user to be measured, the second detecting reference information of the user to be measured including clothing information and/or electronic device carrying information of the user to be measured, the clothing information including at least one of clothing type, clothing thickness, clothing quantity, clothing wearing position, and clothing wearing tightness, and the electronic device carrying information including at least one of electronic device type, electronic device location, and electronic device working state;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured; and
judging whether the detecting affected level is greater than or equal to a preset detecting affected level threshold;
identifying a detecting control parameter corresponding to the preset radar detector according to the first detecting reference information and the second detecting reference information, if a corresponding judging result is positive; and
triggering to perform identifying a detecting control parameter corresponding to the preset radar detector according to the first detecting reference information of the user to be measured, if a corresponding judging result is negative.

As an optional implementation, in the first aspect of the present disclosure, before identifying, according to the signal parameter of the echo signal, biological signal information of the user to be measured, the method further includes:
acquiring a detecting feature parameter of the radar detector when performing the signal detection operation, the detecting feature parameter including at least one of detecting jitter amplitude, detecting jitter frequency, and detecting jitter time;
acquiring an apparatus parameter of the radar detector and identifying, according to the detecting feature parameter and the apparatus parameter, a detection wave affected condition corresponding to the radar detector, the apparatus parameter of the radar detector including apparatus model and/or apparatus loss of the radar detector;
identifying, according to the detection wave affected condition, a detection wave affected level corresponding to the radar detector, and judging whether the detection wave affected level is greater than or equal to a preset detection wave affected level threshold; and,
if a corresponding judging result is positive, identifying, according to the detection wave affected condition, a signal processing parameter of the echo signal, and processing, according to the signal processing parameter, the echo signal to update the echo signal.

Disclosed as a second aspect of the present disclosure is an apparatus of non-contact blood pressure measurement based on radar technology, the apparatus includes:
a detecting module, configured to perform a signal detection operation on a target body part of a user to be measured by means of a preset radar detector to acquire a signal parameter of an echo signal of the user to be measured;
an identifying module, configured to identify, according to the signal parameter of the echo signal, biological signal information of the user to be measured; and
a predicting module, configured to perform a blood pressure prediction operation, according to the biological signal information of the user to be measured, on the user to be measured to acquire a blood pressure parameter of the user to be measured, wherein the blood pressure parameter includes a systolic blood pressure parameter and/or a diastolic blood pressure parameter.

As an optional implementation, in the second aspect of the present disclosure, the signal parameter of the echo signal includes at least one of a waveform increasing rate, a waveform peak, a waveform decreasing rate, a waveform period, a waveform frequency, and a waveform amplitude of the echo signal; the biological signal information of the user to be measured includes at least one of vascular elasticity degree, vascular blood flow rate, vascular blood flow resistance, vascular blood flow volume, and time of change of vascular blood flow of the user to be measured.

the method of the predicting module performing, according to the biological signal information of the user to be measured, a blood pressure prediction operation on the user to be measured to acquire a blood pressure parameter of the user to be measured specifically includes:
identifying, according to the biological signal information of the user to be measured, cardiac feature information of the user to be measured; the cardiac feature information of the user to be measured including cardiac systolic feature information and/or cardiac diastolic feature information of the user to be measured; and
performing, according to the cardiac feature information of the user to be measured, a blood pressure prediction operation on the user to be measured to acquire a blood pressure parameter of the user to be measured.

As an optional implementation, in the second aspect of the present disclosure, the apparatus further includes:
a first acquiring module, configured to, after the predicting module performing, according to the biological signal information of the user to be measured, a blood pressure prediction operation on the user to be measured to acquire a blood pressure parameter of the user to be measured, acquire first target information of the user to be measured, the first target information including basic information and/or health condition information of the user to be measured, the basic information of the user to be measured including at least one of height, weight, gender, and body type of the user to be measured;
the identifying module, further configured to identify, according to the first target information of the user to be measured, a first blood pressure prediction affected condition corresponding to the first target information of the user to be measured, and identify, according to the first blood pressure prediction affected condition corresponding to the first target information of the user to be measured, a first blood pressure prediction affected level corresponding to the first target information of the user to be measured;
a first judging module, configured to judge whether the detecting affected level is greater than or equal to a preset detecting affected level threshold; and
a first correcting module, configured to correct, according to the first target information, the blood pressure parameter of the user to be measured when the judging result of the first judging module is positive.

As an optional implementation, in the second aspect of the present disclosure, the apparatus further includes:
a second acquiring module, configured to, after the predicting module performing, according to the biological signal information of the user to be measured, a blood pressure prediction operation on the user to be measured to acquire a blood pressure parameter of the user to be measured, acquire second target information of the user to be measured, the second target information including at least one of activity state, sleep state, and psychological state of the user to be measured, the activity state including activity intensity and/or activity duration, and the sleep state including sleep depth and/or sleep duration;
the identifying module, further configured to identify, according to the second target information of the user to be measured, a second blood pressure prediction affected condition corresponding to the second target information of the user to be measured, and identify, according to the second blood pressure prediction affected condition corresponding to the second target information of the user to be measured, a second blood pressure prediction affected level corresponding to the second target information of the user to be measured; and
a second judging module, configured to judge whether the second blood pressure prediction affected level is greater than or equal to a preset second affected level threshold;
a second correcting module, configured to correct, according to the second target information, the blood pressure parameter of the user to be measured when the judging result of the second judging module is positive.

As an optional implementation, in the second aspect of the present disclosure, the method of the detecting module performing a signal detection operation on a target body part of a user to be measured by means of a preset radar detector to acquire a signal parameter of an echo signal of the user to be measured specifically includes:
acquiring first detecting reference information of the user to be measured, the first detecting reference information of the user to be measured including health condition and/or current activity of the user to be measured, the current activity including at least one of current activity intensity, current activity posture, current activity amplitude, and duration of activity already performed;
identifying a detecting control parameter corresponding to the preset radar detector according to the first detecting reference information of the user to be measured, the detecting control parameter including at least one of detecting position, detecting distance, detecting wave, detecting duration, and signal receiving position; and
performing, according to the detecting control parameter corresponding to the radar detector, the signal detection operation on the target body part of the user to be measured to acquire the signal parameter of the echo signal of the user to be measured.

As an optional implementation, in the second aspect of the present disclosure, the method of the detecting module performing a signal detection operation on a target body part of a user to be measured by means of a preset radar detector to acquire a signal parameter of an echo signal of the user to be measured specifically further includes:
before identifying a detecting control parameter corresponding to the preset radar detector according to the first detecting reference information of the user to be measured, acquiring second detecting reference information of the user to be measured, the second detecting reference information of the user to be measured including clothing information and/or electronic device carrying information of the user to be measured, the clothing information including at least one of clothing type, clothing thickness, clothing quantity, clothing wearing position, and clothing wearing tightness, and the electronic device carrying information including at least one of electronic device type, electronic device location, and electronic device working state;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured; and
judging whether the detecting affected level is greater than or equal to a preset detecting affected level threshold;
identifying a detecting control parameter corresponding to the preset radar detector according to the first detecting reference information and the second detecting reference information, if a corresponding judging result is positive; and
triggering to perform identifying a detecting control parameter corresponding to the preset radar detector according to the first detecting reference information of the user to be measured, if a corresponding judging result is negative.

As an optional implementation, in the second aspect of the present disclosure, the apparatus further includes:
a third acquiring module, configured to, before identifying, according to the signal parameter of the echo signal, biological signal information of the user to be measured, acquire a detecting feature parameter of the radar detector when performing the signal detection operation, the detecting feature parameter including at least one of detecting jitter amplitude, detecting jitter frequency, and detecting jitter time; and acquire an apparatus parameter of the radar detector, the apparatus parameter of the radar detector including apparatus model and/or apparatus loss of the radar detector;
the identifying module, further configured to identify, according to the detecting feature parameter and the apparatus parameter, a detection wave affected condition corresponding to the radar detector, and identify, according to the detection wave affected condition, a detection wave affected level corresponding to the radar detector;
a third judging module, configured to judge whether the detection wave affected level is greater than or equal to a preset detection wave affected level threshold;
the identifying module, further configured to, if a corresponding judging result of the third judging module is positive, identify, according to the detection wave affected condition, a signal processing parameter of the echo signal, and process, according to the signal processing parameter, the echo signal to update the echo signal.

Disclosed as a third aspect in the present disclosure is an apparatus of non-contact blood pressure measurement based on radar technology, and the apparatus includes:
a memory, memorized with an executable code; and
a processor, coupled with the memory.

The processor invokes the executable code memorized in the memory to perform the method of non-contact blood pressure measurement based on radar technology disclosed by the first aspect of the present disclosure.

Disclosed as a fourth aspect in the present disclosure is a non-transitory computer memory medium, and the non-transitory computer memory medium memorizes computer instructions; when the computer instructions are invoked, the method of non-contact blood pressure measurement based on radar technology disclosed in the first aspect of the present disclosure is performed.

Compared to the prior art, the embodiments of the present disclosure have beneficial effects as follows.

In the embodiments of the present disclosure, it is performed a signal detection operation on a target body part of a user to be measured by means of a radar detector to acquire a signal parameter of an echo signal of the user to be measured; it is identified, according to the signal parameter of the echo signal, biological signal information of the user to be measured; and it is performed a blood pressure prediction operation, according to the biological signal information of the user to be measured, on the user to be measured so as to acquire a blood pressure parameter of the user to be measured. It is evident that the implementation of the present diclosure is capable of acquiring, by means of a radar detector, an echo signal of the user to be measured, and acquiring a blood pressure parameter of the user to be measured by performing, according to the echo signal, a blood pressure prediction on the user to be measured. In such a method, the blood pressure is measured in a non-contact manner, which improves the flexibility of the blood pressure measurement of the subject, which in turn improves the efficiency and accuracy of the blood pressure measurement of the subject, and reduces the discomfort of the measurement of the subject, which in turn improves the device use experience of the subject.

### BRIEF DESCRIPTION OF THE DRAWINGS

In order to illustrate the technical solutions of the embodiments of the present disclosure more clearly, the following drawings are briefly described as required in the context of the embodiments. Obviously, the following drawings illustrate only some of the embodiments of the present disclosure. Other relevant drawings may be obtained on the basis of these drawings without any creative effort by those skilled in the art.

FIG. 1 is a schematic diagram of a scenario of non-contact blood pressure measurement based on radar technology disclosed in an embodiment of the present disclosure;

FIG. 2 is a schematic flow diagram of a method of non-contact blood pressure measurement based on radar technology disclosed in an embodiment of the present disclosure.

FIG. 3 is a schematic flow diagram of another method of non-contact blood pressure measurement based on radar technology disclosed in an embodiment of the present disclosure.

FIG. 4 is a schematic structural diagram of an apparatus of non-contact blood pressure measurement based on radar technology disclosed in an embodiment of the present disclosure.

FIG. 5 is a schematic structural diagram of another apparatus of non-contact blood pressure measurement based on radar technology disclosed in an embodiment of the present disclosure.

FIG. 6 is a schematic structural diagram of yet another apparatus of non-contact blood pressure measurement based on radar technology disclosed in an embodiment of the present disclosure.

FIG. 7 is a schematic diagram of a prediction model framework employed by the method of non-contact blood pressure measurement disclosed in an embodiment of the present disclosure;

FIG. 8 is a schematic diagram of a network framework for a prediction model disclosed in an embodiment of the present disclosure.

### DETAILED DESCRIPTION

For a better understanding of the solutions of the present disclosure by those skilled in the art, the technical solutions in the embodiments of the present disclosure are clearly and completely described and discussed below in conjunction with the attached drawings of the embodiments of the present disclosure. Obviously, the embodiments described herein are only some of the embodiments of the present disclosure but not all of them. Based on the embodiments in the present disclosure, all other embodiments acquired by those skilled in the art without inventive effort fall within the scope of protection of the present disclosure.

The terms “first”, “second”, and the like in the specification, the claims and the above-mentioned drawings of the present disclosure are used to identify different objects and are not intended to describe a particular sequence. Additionally, the terms “comprise” and “include”, and any derivatives and conjugations thereof, are intended to cover non-exclusive inclusion. For example, a process, method, apparatus, product, or device that includes a series of steps or units is not limited to the listed steps or units, but optionally also includes steps or units that are not listed, or optionally also includes other steps or units that are inherent to those processes, methods, products, or devices.

The term “embodiment” herein means that a particular feature, structure or characteristic described in conjunction with an embodiment may be included in at least one embodiment of the present disclosure. The presence of the term in various places in the specification does not necessarily indicate the same embodiment, nor is it a separate or alternative embodiment that is mutually exclusive with other embodiments. It is understood, both explicitly and implicitly, by those skilled in the art that the embodiments described herein may be combined with other embodiments.

Disclosed in the present disclosure is a method and an apparatus of non-contact blood pressure measurement based on radar technology, so that blood pressure can be measured in a non-contact manner, which improves the flexibility of the blood pressure measurement of the subject, which in turn improves the efficiency and accuracy of the blood pressure measurement of the subject, and reduces the discomfort of the measurement of the subject, which in turn improves the device use experience of the subject.

### First Embodiment

Referring to FIG. 2, FIG. 2 is a schematic flow diagram of the method of non-contact blood pressure measurement based on radar technology disclosed in the present embodiment of the disclosure. Optionally, the method may be implemented by a blood pressure measurement device, which may be integrated in the radar detector or may exist independently of the radar detector, or may be a local server or a cloud server, configured to process the non-contact blood pressure measurement process based on the radar technology, or the like, which is not limited by the embodiments of the present disclosure. As shown in FIG. 2, the method of non-contact blood pressure measurement based on radar technology may include the following steps:
In step 101, a signal detection operation is performed on a target body part of a user to be measured by means of a preset radar detector to acquire a signal parameter of an echo signal of the user to be measured;

In the present embodiment of the disclosure, optionally, the target body part may be such as chest, neck, and wrists.

Further and optionally, the signal parameter of the echo signal includes at least one of a waveform increasing rate, a waveform peak, a waveform decreasing rate, a waveform period, a waveform frequency, and a waveform amplitude of the echo signal.

In step 102, biological signal information of the user to be measured is identified according to the signal parameter of the echo signal.

In the present embodiment of the disclosure, optionally, the biological signal information of the user to be measured includes at least one of vascular elasticity degree, vascular blood flow rate, vascular blood flow resistance, vascular blood flow volume, and time of change of vascular blood flow of the user to be measured.

In step 103, according to the biological signal information of the user to be measured, a blood pressure prediction operation on the user to be measured is performed to acquire a blood pressure parameter of the user to be measured.

In the present embodiment of the disclosure, optionally, the blood pressure parameter includes a systolic blood pressure parameter and/or a diastolic blood pressure parameter.

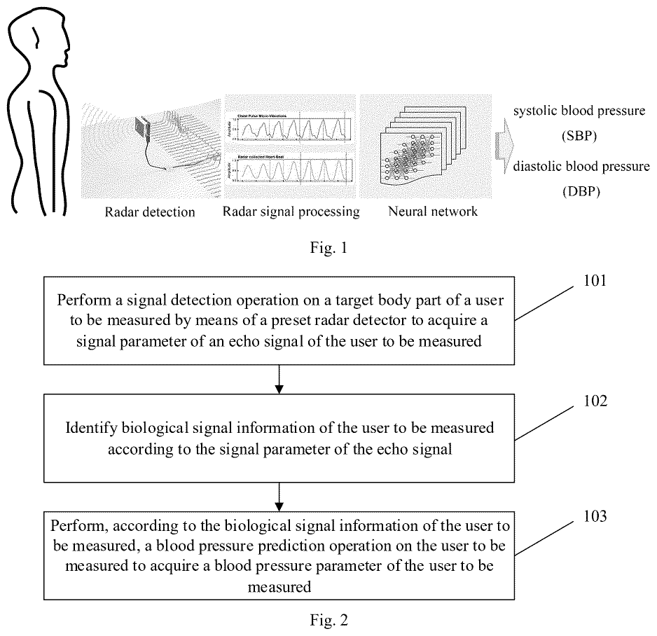

For example, as shown in FIG. 1, FIG. 1 is a schematic diagram of a scenario of non-contact blood pressure measurement based on radar technology disclosed by the present embodiment of the disclosure. A specific microwave signal is first emitted to the chest of the user to be measured using a radar detector. When these incoming signals encounter the chest, the echo is reflected to achieve the chest signal detection of the target, and then by means of a preset prediction model (as shown in FIG. 7, there are two main modules in the blood pressure measurement device: one is the radar signal processing DSP module, the other is the inference AI framework for blood pressure prediction; the radar module may employ FMWC technology, mainly since FMWC can measure the distance and speed of the target at the same time, and has a mature and fast data signal processing chain), and according to the signal parameters of the echo signal, the biological signal information of the user to be measured is identified (the prediction model can analyze the echo signal in detail) to predict the blood pressure parameters of the user to be measured according to the biological signal information. It should be noted that the principle of the aforementioned implementation process is that the chest of the user to be measured causes subtle displacement changes on the surface of the chest cavity due to the physiological movement of the internal heart. These changes modulate the incident signal of the radar. Therefore, the echo signal generated from the chest carries the heart movement information of the user to be measured.

It is evident that the implementation of the present embodiment of the disclosure is capable of acquiring, by means of a radar detector, an echo signal of the user to be measured, and acquiring a blood pressure parameter of the user to be measured by performing, according to the echo signal, a blood pressure prediction on the user to be measured. In such a method, the blood pressure is measured in a non-contact manner, which improves the flexibility of the blood pressure measurement of the subject, which in turn improves the efficiency and accuracy of the blood pressure measurement of the subject, and reduces the discomfort of the measurement of the subject, which in turn improves the device use experience of the subject.

In an optional implementation, after aforementioned step 103 in which a blood pressure prediction operation is performed on the user to be measured according to the biological signal information of the user to be measured to acquire a blood pressure parameter of the user to be measured, the method further includes:
acquiring first target information of the user to be measured;
identifying, according to the first target information of the user to be measured, a first blood pressure prediction affected condition corresponding to the first target information of the user to be measured, and identifying, according to the first blood pressure prediction affected condition corresponding to the first target information of the user to be measured, a first blood pressure prediction affected level corresponding to the first target information of the user to be measured; and
judging whether the first blood pressure prediction affected level is greater than or equal to a preset first affected level threshold, and if yes, correcting the blood pressure parameter of the user to be measured according to the first target information.

In this optional implementation, optionally, the first target information includes basic information and/or health condition information of the user to be measured, and the basic information of the user to be measured includes at least one of height, weight, gender, and body type of the user to be measured;

Further, when it is judged that the first blood pressure prediction affected level is less than the preset first affected level threshold, the blood pressure parameters do not need to be corrected, and the predicted blood pressure parameters are directly employed.

For example, if the current respiration rate (one of the health status information) of the user to be measured is high, but his respiration rate is in a normal range when he is actually sitting, the predicted blood pressure parameter may be high. In this case, it can be determined that the first blood pressure prediction affected level is greater than or equal to the preset first affected level threshold, so as to correct the blood pressure parameter of the user to be measured according to his current respiration rate.

It is evident that, in the optional embodiment, it may be identified the corresponding first blood pressure prediction affected condition according to the first target information of the user to be measured, and then judged whether it is necessary to further correct the blood pressure parameters of the user to be measured. In this case, it is conducive to improving the correction reliability and accuracy of the blood pressure parameters of the user to be measured, and further conducive to accurately predicting the blood pressure parameters of the user to be measured, thereby improving the accuracy of the health status assessment of the user to be measured subsequently.

In another optional implementation, after aforementioned step 103 in which a blood pressure prediction operation is performed on the user to be measured according to the biological signal information of the user to be measured to acquire a blood pressure parameter of the user to be measured, the method further includes:
acquiring second target information of the user to be measured;
identifying, according to the second target information of the user to be measured, a second blood pressure prediction affected condition corresponding to the second target information of the user to be measured, and identifying, according to the second blood pressure prediction affected condition corresponding to the second target information of the user to be measured, a second blood pressure prediction affected level corresponding to the second target information of the user to be measured; and
judging whether the second blood pressure prediction affected level is greater than or equal to a preset second affected level threshold, and if yes, correcting the blood pressure parameter of the user to be measured according to the second target information.

In this optional implementation, optionally, the second target information includes at least one of activity state, sleep state, and psychological state of the user to be measured, the activity state includes activity intensity and/or activity duration, and the sleep state includes sleep depth and/or sleep duration;

Further, when it is judged that the second blood pressure prediction affected level is less than the preset second affected level threshold, the blood pressure parameters do not need to be corrected, and the predicted blood pressure parameters are directly employed.

For example, if the user to be measured is currently in a nervous mood, so that the number of heart beats rises, the predicted blood pressure parameter may be high. In this case, it can be determined that the second blood pressure prediction affected level is greater than or equal to the preset second affected level threshold, so as to correct the blood pressure parameter of the user to be measured according to his psychological state information.

It should be noted that, as shown in FIG. 7, the following factors should be considered in the prediction model framework module: 1) high-dimensional fine feature extraction of dynamic biological signals; 2) reasoning generalization ability and reasoning range of standard blood pressure models; 3) special populations or special body types or body types that change over time. According to the above requirements, the prediction model framework may include these two basic logical functions: the blood pressure prediction sub-model F (for blood pressure prediction) and the system correction sub-model g (for blood pressure correction).

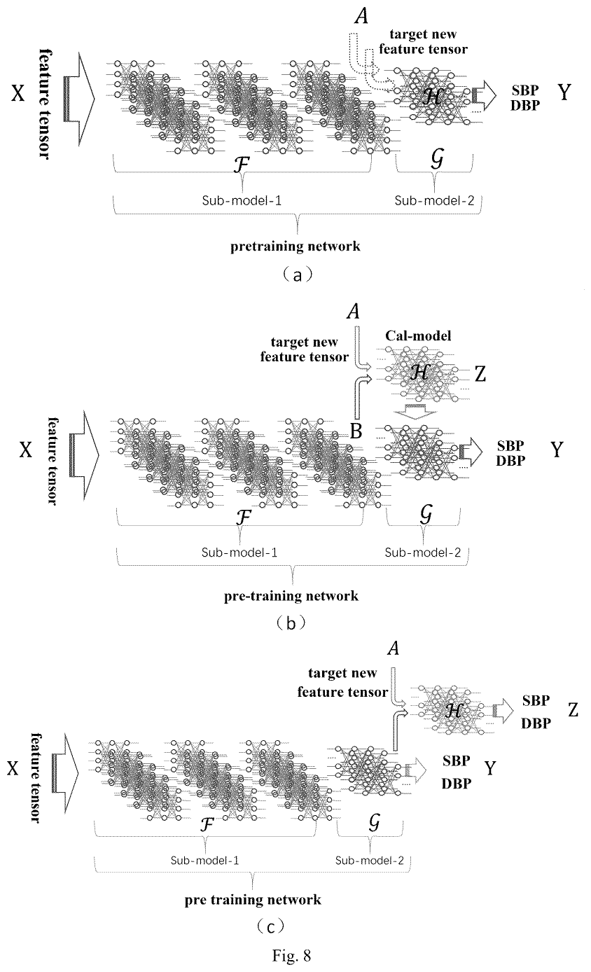

Specifically, the network framework included in the prediction model can be implemented in a variety of ways as shown in the inset (a)-(c) of FIG. 8. Among them, the symbols in FIG. 8 have the following meanings: X: feature tensor, which is the output of the radar signal processing unit; Y: prediction signal vector of the prediction model, including systolic and diastolic blood pressure; A: new feature tensor that the user can input, such as the aforementioned first target information and/or second target information; B: intermediate state tensor of the sub-model output; F: first sub-model, configured to predict blood pressure, the parameters are fixed and are no longer adjusted after the model is trained; g: second sub-model, configured to correct blood pressure, and the pre-trained parameters can be fine-tuned a second time.

The first method is shown in the inset (a) of FIG. 8: the entire blood pressure prediction equation is as follows: Y=g (F (X), A), and in this network framework, g=H. The basic criterion for the division of F and g is that g is a sub-model consisting of the last few layers of the whole model, and the trainable parameter set of this sub-model is relatively small. This division of functions should take full account of the storage and computing power of the deployed hardware when pre-training the model. Therefore, the network framework has the following advantages: 1) When pre-training the model, the dimension of the input signal on the radar side and the existence and computing power of the subsequent hardware deployment can be fully considered, and a sub-model based on globalization can be designed to be optimized a second time to adapt to the special constitution of the non-measurable target, and continue to correct and compensate for the global performance to achieve more accurate blood pressure measurement; 2) The sub-model g can achieve a variety of functions and requires less resources when deployed, such as combining the sub-model F to achieve blood pressure prediction, and using new feature inputs to achieve offline correction of system performance.

The second method is shown in the inset (b) of FIG. 8: when the pre-trained model is deployed, an additional correction model H needs to be deployed, in which the correction sub-model H and the sub-model g have the same structure. The system includes a blood pressure prediction sub-model and a system correction sub-model, and the two sub-models can work in parallel, in which the blood pressure prediction equation is: Y=g (F (X)), and the system correction variance equation is: Z=H (A, B)=H (A, F (X)), in this network framework, H=g, and the g parameter can be optimized a second time. The basic criteria for the division of F and g can be described with reference to the network framework in the inset (a) of FIG. 8, but the main difference from the network framework in the inset (a) of FIG. 8 is that the system correction sub-model H is an entity unit that requires simultaneous deployment of the pre-trained network. Therefore, the network framework has the following advantages: 1) The system supports two tasks that can be run in parallel, that is, when the prediction model predicts the target blood pressure, the system function correction work can be carried out at the same time, and the two tasks are independent of each other; 2) The sub-model H can be regarded as a twin model of the sub-model g. After the system function correction work is completed, the g model parameters can be quickly updated, and the online correction function can be realized to achieve more accurate blood pressure measurement; 3) The sub-model H does not participate in the normal blood pressure measurement work, so the resources allocated to the system can be dynamically optimized.

The third method is shown in the inset (c) of FIG. 8: When the pre-trained model is deployed, an additional correction model H needs to be deployed, in which the correction sub-model H and the sub-model g can have different structures. The system includes a blood pressure prediction sub-model and a system correction sub-model. The two sub-models work in a serial manner, in which the blood pressure prediction equation is: Z=H (A, Y)=H (A, g (F (X))). In this network framework, H ≠g, and the parameters of the pre-trained model consisting of F and g are fixed, and a secondary optimization is not allowed to be performed. Compared with the previous two network frameworks, the main difference of this network framework is that the correction sub-model H may be a sub-model with fewer parameters and smaller scale, and the pre-trained network needs to be deployed at the same time. Therefore, the network framework has the following advantages: 1) The design of the system correction sub-model H is flexible and can be different from the sub-model g, which can be a sub-model with fewer parameters and smaller scale; 2) The pre-trained model {F, g} can be used as a data preprocessing module of the sub-model H, and the data dimension of the sub-module H input can be seen to be small, so the sub-module can be easily deployed on the terminal-side device; 3) Based on the small amount of data interaction between {F, g} and H, the two functional units can be deployed in a divided manner; 4) The sub-model H can achieve offline correction function and achieve more accurate blood pressure measurement.

Further, the meaning of the representation of the intermediate state tensor of the model is closely related to the specific structure of the model. Taking the transformer structure as an example, the intermediate state information includes the following information:

### 1. Signal Characterization

Time series feature extraction: Encoder intermediate layer output is a feature representation of the time series of the input radar signal. It can automatically extract a variety of meaningful features from the original radar signal, such as the periodicity and trend of the signal, by means of self-attention mechanisms and other operations. These characteristics are critical for understanding the underlying patterns of radar signals and predicting their future direction.

Multi-scale feature capture: Different intermediate layers can capture features at different scales. Shallower intermediate layers may focus on extracting localized short-term features, while deeper intermediate layers are able to capture more global, longer-term features. By means of this multi-level feature extraction, Transformer is able to comprehensively understand the complex structure of the radar signal, thus providing richer information for subsequent predictions.

### 2. Signal Semantic Information

Physical meaning understanding: Encoder intermediate layer output also contains some “semantic” information of the radar signal, that is, the physical meaning represented by the signal and the relevant information in the actual scene. For example, the characteristics such as the strength and frequency of the radar signal are closely related to the physical properties such as the distance and speed of the target. The feature representation output by the intermediate layer of Encoder may include the correlation and potential laws between these physical properties, so as to achieve a deeper understanding of the radar signal and modeling at the semantic level.

Contextual information fusion: The output of each intermediate layer fuses the contextual information of the input radar signal. The self-attention mechanism enables the model to focus on the relationships between different locations in the signal sequence, thereby integrating global contextual information into the feature representation of each location. This fusion of contextual information contributes to a better understanding of radar signal trends and interdependencies, improving prediction accuracy.

### 3. Data Compression and Abstraction

Dimension reduction representation: Encoder intermediate layer output is a compressed and abstract representation of input radar signal data. As the number of network layers is increased, the feature dimension gradually is decreased, but at the same time retains the most critical and representative information. This dimension reduction representation not only reduces the amount of data storage and computation, but also removes noise and redundant information, highlighting the main features and laws of the signal, making the model easier to learn and process.

Common feature extraction: In the process of compression and abstraction, the Encoder intermediate layer may extract common features between different radar signal data. These common characteristics reflect the general laws and patterns of radar signals in general, and help the model to generalize and predict radar signals in different scenarios. By learning these common characteristics, the model can better cope with various changes and uncertainties, and improve the stability and reliability of forecasts.

### 4. Potential Target Information

Target characteristic reflection: Since the radar signal is closely related to the state and characteristics of the target, the Encoder intermediate layer output also indirectly reflects some information of the target. For example, features such as the motion state, size, and shape of the target may leave corresponding traces in the feature representation of the radar signal. Through the analysis and mining of the output of the intermediate layer, the feature information related to the target can be extracted to provide support for tasks such as target identification, tracking, and classification.

Target relationship modeling: For radar signals from multiple targets, the Encoder intermediate layer output can also model the relationship between the targets. The self-attention mechanism can capture the interaction and correlation between the radar signals of different targets, so that the relative position, speed difference, coordinated motion and other relationship information between the targets can be reflected in the characteristic representation. This is of great significance for radar signal processing and analysis in multi-target scenarios.

It is evident that, in the optional embodiment, it is identified the corresponding second blood pressure prediction affected condition according to the second target information of the user to be measured, and then judged whether it is necessary to further correct the blood pressure parameters of the user to be measured. In this case, the judgment comprehensiveness and accuracy of the blood pressure condition of the user to be measured can be improved, and then the blood pressure prediction deviation caused by the current state of the user to be measured and/or different self conditions can be effectively corrected, thereby improving the reliability and accuracy of blood pressure prediction. At the same time, in the network framework design, by rationally dividing the blood pressure prediction sub-model and the system correction sub-model, both the limitations of hardware storage and computing capacity are considered, and the optimal division of functions is achieved. This helps to reduce the cost and complexity of hardware deployment while maintaining the prediction accuracy.

### Second Embodiment

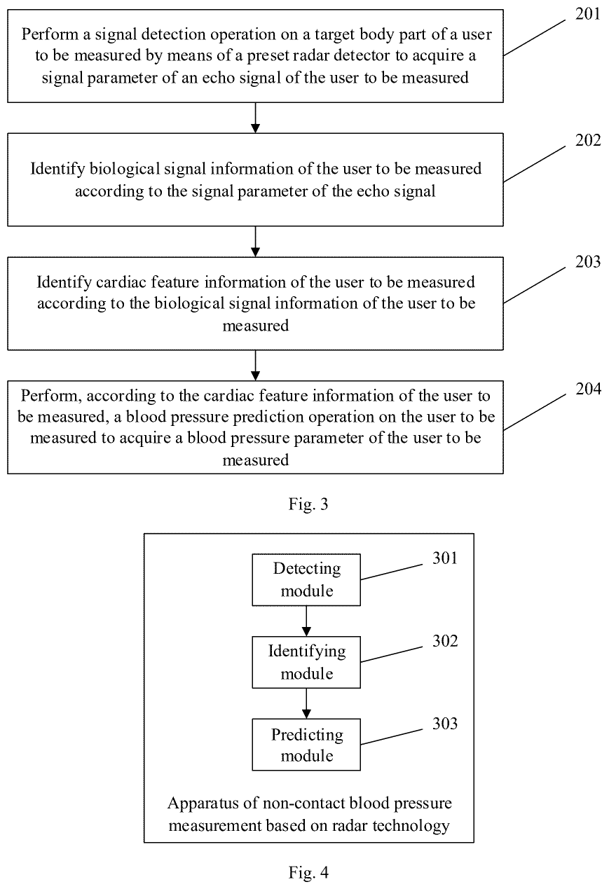

Referring to FIG. 3, FIG. 3 is a schematic flow diagram of another method of non-contact blood pressure measurement based on radar technology disclosed in the present embodiment of the disclosure. Optionally, the method may be implemented by a blood pressure measurement device, which may be integrated in the radar detector or may exist independently of the radar detector, or may be a local server or a cloud server, configured to process the non-contact blood pressure measurement process based on the radar technology, or the like, which is not limited by the embodiments of the present disclosure. As shown in FIG. 3, the method of non-contact blood pressure measurement based on radar technology may include the following steps:

In step 201, a signal detection operation on a target body part of a user to be measured is performed by means of a preset radar detector to acquire a signal parameter of an echo signal of the user to be measured.

In step 202, biological signal information of the user to be measured is identified according to the signal parameter of the echo signal.

In step 203, cardiac feature information of the user to be measured is identified according to the biological signal information of the user to be measured.

In the present embodiment of the disclosure, optionally, the cardiac feature information of the user to be measured includes cardiac systolic feature information and/or cardiac diastolic feature information of the user to be measured; and

It should be noted that when the heart performs contraction and diastolic activities, it produces a pressure change in the blood, which propagates along the arterial vessels in the form of waves, forming a cardiac micro-motion signal. It reflects the physiological characteristics such as the periodic activity of the heart and the elasticity of the blood vessels. It is one of the important characteristics of the physiological state of the cardiovascular system. Therefore, the cardiac micro-motion signal is a relatively complex biological signal containing rich physiological information. Among them, the biological signal mainly includes the following:

### 1. Morphological Characteristics of Pressure Waves

Ascending branch: the systolic phase of the heart; the ventricles ejaculate rapidly and the blood flows rapidly into the arteries, causing a sharp rise in intra-arterial pressure and the formation of an ascending branch of the pressure wave. Its slope and amplitude can reflect the strength and velocity of ventricular contraction.
Peak: the highest point of the pressure wave, corresponding to the highest pressure of the ventricular systolic period, commonly referred to as systolic blood pressure. The size of the systolic blood pressure is related to the pumping function of the heart, vascular resistance and other factors.

Descending branch: In the late ventricular systole, the ejection speed gradually slows down and the arterial pressure begins to drop, forming the descending branch of the pressure wave. The shape of the descending branches is complex, including multiple peaks and troughs, some of which are related to the physiological processes of the heart and blood vessels.

Repetition waves: On the descending branch, there is sometimes an obvious wavelet peak called a repetition wave. It is caused by the rebound and vibration of blood within the arteries after the aortic valve is closed, and its position and amplitude can reflect the elasticity and peripheral resistance of the blood vessels.

### 2. Time Interval Information

Cardiac cycle: The time elapsed by a single systole and diastole of the heart, determined by measuring the time interval between the start points of two adjacent pressure waves. The length of the cardiac cycle is closely related to the heart rate, and the faster the heart rate, the shorter the cardiac cycle.

Systolic period: The time from the start of the pressure wave rise to the peak, representing the duration of ventricular contraction. The length of the systolic period can reflect the state of ventricular systolic function, for example, the systolic period may be shortened when the myocardial systolic force is enhanced.

Diastolic period: The time, from the peak of the pressure wave to the next start point of the ascending branch, is the duration of ventricular diastole. Diastolic period is important for the filling of the heart and perfusion of the coronary arteries, and abnormal changes may be related to diseases such as cardiac diastolic dysfunction.

Frequency characteristics: The frequency characteristics of the cardiac micro-motion signal can be obtained by spectrum analysis. The frequency component of the cardiac micro-motion signal is mainly concentrated in the low frequency band, which is related to the fundamental frequency of the heart rate and its harmonics. In addition, it may also contain some high-frequency components, which may be related to factors such as elastic vibration of blood vessels, turbulence of blood, and other factors. By analyzing frequency characteristics, dynamic information about the heart and vascular system, such as changes in vascular compliance, can be obtained.

Amplitude characteristics: The amplitude of the pressure wave reflects the magnitude of the blood pressure and, in addition to the systolic pressure mentioned above, includes diastolic pressure, the lowest pressure within the ventricular end-diastolic artery. Pulse pressure is the difference between systolic and diastolic blood pressure, which reflects the amplitude of fluctuations in arterial blood pressure, and is related to factors such as cardiac stroke output and vascular elasticity. In addition, the change in the amplitude of the micro-motion signal can also reflect the rhythm of cardiac contraction and relaxation and the regulation function of blood vessels.

In step 204, according to the cardiac feature information of the user to be measured, a blood pressure prediction operation on the user to be measured is performed to acquire a blood pressure parameter of the user to be measured.

In the present embodiment of the disclosure, regarding other descriptions of step 201 to step 202, please refer to the other detailed descriptions in the first embodiment regarding step 101 to step 102, which is not repeated in the present embodiment of the disclosure.

It is evident that, by implementing the present embodiment of the disclosure, it is identified the biological signal information of the user to be measured according to the signal parameters of the echo signal of the user to be measured obtained by the radar detector, and then identified the cardiac feature information of the user to be measured, so as to realize the non-contact blood pressure measurement process of the user to be measured. In this case, it can provide rich data support for blood pressure prediction, realize the high-precision prediction process of blood pressure parameters, and improve the accuracy of blood pressure monitoring. In addition, it can also improve the blood pressure measurement comfort of the user to be measured, and reduce the skin discomfort or infection risk caused by improper sensor contact or long-term wear.

In an optional implementation, in the aforementioned step 201, the signal detection operation being performed on a target body part of a user to be measured by means of a preset radar detector to acquire a signal parameter of an echo signal of the user to be measured includes:
acquiring first detecting reference information of the user to be measured;
identifying, according to the first detecting reference information of the user to be measured, a detecting control parameter corresponding to the preset radar detector,
performing, according to the detecting control parameter corresponding to the radar detector, the signal detection operation on the target body part of the user to be measured to acquire the signal parameter of the echo signal of the user to be measured.

In this optional implementation, optionally, the first detecting reference information of the user to be measured includes health condition and/or current activity of the user to be measured, and the current activity includes at least one of current activity intensity, current activity posture, current activity amplitude, and duration of activity already performed. Further and optionally, the detecting control parameter includes at least one of detecting position, detecting distance, detecting wave, detecting duration, and signal receiving position.

For example, since the user to be measured is in a highly active state, such as the chest is rotated left and right, the radar detector may need to dynamically transmit microwave signals to the user to be measured, or dynamically collect the echo signals of the user to be measured.

It is evident that, by implementing the optional embodiment, it is identified the detecting control parameters corresponding to the radar detector based on the first detecting reference information of the user to be measured, and then perform signal detection on the target body part of the user to be measured, thus improving the control flexibility and accuracy of the radar detector, thereby improving the microwave signal transmission accuracy for the user to be measured and the echo signal reception accuracy for the user to be measured, thereby facilitating the subsequent accurate blood pressure prediction process.

In another optional implementation, before the aforementioned steps of identifying a detecting control parameter corresponding to the preset radar detector according to the first detecting reference information of the user to be measured, the method further includes:
acquiring second detecting reference information of the user to be measured;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured; and
judging whether the detecting affected level is greater than or equal to a preset detecting affected level threshold;
identifying a detecting control parameter corresponding to the preset radar detector according to the first detecting reference information and the second detecting reference information if a corresponding judging result is positive; and
triggering to perform identifying a detecting control parameter corresponding to the preset radar detector according to the first detecting reference information of the user to be measured if a corresponding judging result is negative.

In this optional implementation, optionally, the second detecting reference information of the user to be measured includes clothing information and/or electronic device carrying information of the user to be measured, the clothing information includes at least one of clothing type, clothing thickness, clothing quantity, clothing wearing position, and clothing wearing tightness, and the electronic device carrying information includes at least one of electronic device type, electronic device location, and electronic device working state; For example, since the clothes worn by the user to be measured are too thick and a smartphone is carried, the strength of the echo signal received by the radar detector may be weakened, and the frequency and amplitude of the echo signal may be disturbed. Therefore, it is necessary to further consider the detection influence caused by the second detection reference information to accurately identify the detection control parameters corresponding to the radar detector.

It is evident that, by implementing the optional embodiment, it is identified further the detecting control parameters corresponding to the radar detector based on the second detecting reference information of the user to be measured, and then perform signal detection on the target body part of the user to be measured, thus further improving the control flexibility and accuracy of the radar detector, thereby is conducive to improving the microwave signal transmission accuracy for the user to be measured and the echo signal reception accuracy for the user to be measured, thereby facilitating the subsequent quick and accurate blood pressure prediction process.

In yet another optional implementation, in the aforementioned step 202, before identifying, according to the signal parameter of the echo signal, biological signal information of the user to be measured, the method further includes:
acquiring a detecting feature parameter of the radar detector when performing the signal detection operation;
acquiring an apparatus parameter of the radar detector and identifying, according to the detecting feature parameter and the apparatus parameter, a detection wave affected condition corresponding to the radar detector;
identifying, according to the detection wave affected condition, a detection wave affected level corresponding to the radar detector, and judging whether the detection wave affected level is greater than or equal to a preset detection wave affected level threshold; and,
if a corresponding judging result is positive, identifying, according to the detection wave affected condition, a signal processing parameter of the echo signal, and processing, according to the signal processing parameter, the echo signal to update the echo signal.

In this optional implementation, optionally, the detecting feature parameter includes at least one of detecting jitter amplitude, detecting jitter frequency, and detecting jitter time. Further and optionally, the apparatus parameter of the radar detector includes apparatus model and/or apparatus loss of the radar detector. For example, when a hand-held radar detector is required to detect the signal on the chest of the user to be measured, the radar detector may have a jitter condition during this process. In this case, it is necessary to identify the sensitivity of the radar detector to this jitter condition in combination with the model parameters and loss parameters of the radar detector. If the sensitivity is large (that is, the detection wave is affected to a large extent), it is necessary to identify the signal processing parameters of the echo signal, such as de-noising, de-differentiation, de-trending, and band-pass filtering, so as to process the echo signal.

It should be noted that after signal processing, a relatively clean and effective signal that responds to cardiac micro-motion information can be extracted. These processed signals can be one-dimensional time series information reflecting a specific physiological characteristic information (such as blood flow velocity and blood flow resistance); or processed signals are a high latitude time series information representing a variety of specific physiological characteristics information at a certain time or period of time.

It is evident that, by implementing the optional embodiment, it is identified the corresponding detection wave affected condition according to the detection feature parameters and apparatus parameters of the radar detector, such as wave amplitude change and wave velocity change. Then, when the detection wave is greatly affected, the echo signal is processed according to the detection wave affected condition, which is conducive to improving the reliability and accuracy of the collected echo signal, thereby improving the analysis reliability and accuracy of the subsequent echo signal, and thus improving the efficiency and accuracy of the blood pressure measurement for the user to be measured.

### Third Embodiment

Referring to FIG. 4, FIG. 4 is a schematic structural diagram of an apparatus of non-contact blood pressure measurement based on radar technology disclosed in the present embodiment of the disclosure. As shown in FIG. 4, the apparatus of non-contact blood pressure measurement based on radar technology may include:
a detecting module 301, configured to perform a signal detection operation on a target body part of a user to be measured by means of a preset radar detector to acquire a signal parameter of an echo signal of the user to be measured;
an identifying module 302, configured to identify biological signal information of the user to be measured according to the signal parameter of the echo signal; and
a predicting module 303, configured to perform a blood pressure prediction operation on the user to be measured according to the biological signal information of the user to be measured to acquire a blood pressure parameter of the user to be measured.

In the embodiment of the present disclosure, the blood pressure parameter includes a systolic blood pressure parameter and/or a diastolic blood pressure parameter.

It is evident that the implementation of the apparatus of non-contact blood pressure measurement based on radar technology described in FIG. 4 is capable of acquiring, by means of a radar detector, an echo signal of the user to be measured, and acquiring a blood pressure parameter of the user to be measured by performing, according to the echo signal, a blood pressure prediction on the user to be measured. In such a method, the blood pressure is measured in a non-contact manner, which improves the flexibility of the blood pressure measurement of the subject, which in turn improves the efficiency and accuracy of the blood pressure measurement of the subject, and reduces the discomfort of the measurement of the subject, which in turn improves the device use experience of the subject.

In an optional implementation, the signal parameter of the echo signal includes at least one of a waveform increasing rate, a waveform peak, a waveform decreasing rate, a waveform period, a waveform frequency, and a waveform amplitude of the echo signal; the biological signal information of the user to be measured includes at least one of vascular elasticity degree, vascular blood flow rate, vascular blood flow resistance, vascular blood flow volume, and time of change of vascular blood flow of the user to be measured.

The method of the predicting module 303 performing a blood pressure prediction operation on the user to be measured according to the biological signal information of the user to be measured to acquire a blood pressure parameter of the user to be measured specifically includes:
identifying, according to the biological signal information of the user to be measured, cardiac feature information of the user to be measured;
performing, according to the cardiac feature information of the user to be measured, a blood pressure prediction operation on the user to be measured to acquire a blood pressure parameter of the user to be measured.

In this optional implementation, the cardiac feature information of the user to be measured includes cardiac systolic feature information and/or cardiac diastolic feature information of the user to be measured.

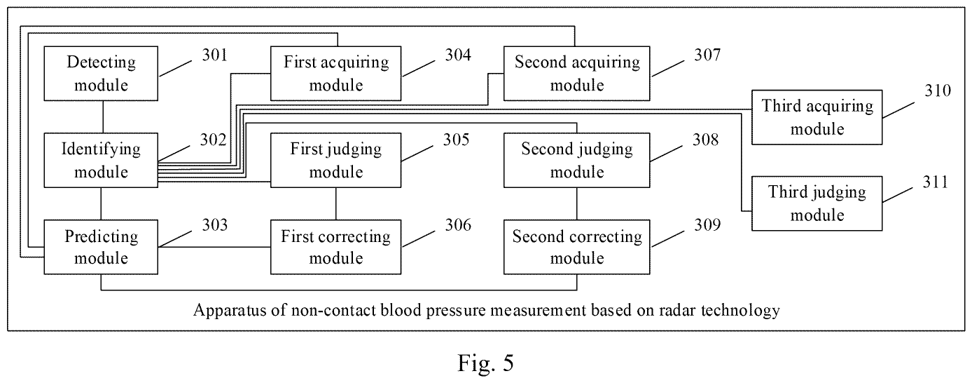

It is evident that, by implementing the apparatus of non-contact blood pressure measurement based on radar technology described in FIG. 5, it is identified the biological signal information of the user to be measured according to the signal parameters of the echo signal of the user to be measured obtained by the radar detector, and then identified the cardiac feature information of the user to be measured, so as to realize the non-contact blood pressure measurement process of the user to be measured. In this case, it can provide rich data support for blood pressure prediction, realize the high-precision prediction process of blood pressure parameters, and improve the accuracy of blood pressure monitoring. In addition, it can also improve the blood pressure measurement comfort of the user to be measured, and reduce the skin discomfort or infection risk caused by improper sensor contact or long-term wear.

In another optional implementation, the apparatus further includes:
a first acquiring module 304, configured to, after the predicting module 303 performs a blood pressure prediction operation on the user to be measured according to the biological signal information of the user to be measured to acquire a blood pressure parameter of the user to be measured, acquire first target information of the user to be measured;
the identifying module 302, further configured to identify, according to the first target information of the user to be measured, a first blood pressure prediction affected condition corresponding to the first target information of the user to be measured, and identify, according to the first blood pressure prediction affected condition corresponding to the first target information of the user to be measured, a first blood pressure prediction affected level corresponding to the first target information of the user to be measured; and
a first judging module 305, configured to judge whether the detecting affected level is greater than or equal to a preset detecting affected level threshold;
a first correcting module 306, configured to correct the blood pressure parameter of the user to be measured according to the first target information if the judging result of the first judging module 305 is positive.

In this optional implementation, the first target information includes basic information and/or health condition information of the user to be measured, and the basic information of the user to be measured includes at least one of height, weight, gender, and body type of the user to be measured.

It is evident that, by implementing the apparatus of non-contact blood pressure measurement based on radar technology described in FIG. 5, it may be identified the corresponding first blood pressure prediction affected condition according to the first target information of the user to be measured, and then judged whether it is necessary to further correct the blood pressure parameters of the user to be measured. In this case, it is conducive to improving the correction reliability and accuracy of the blood pressure parameters of the user to be measured, and further conducive to accurately predicting the blood pressure parameters of the user to be measured, thereby improving the accuracy of the health status assessment of the user to be measured subsequently.

In another optional implementation, the apparatus further includes:
a second acquiring module 307, configured to, after the predicting module 303 performs a blood pressure prediction operation on the user to be measured according to the biological signal information of the user to be measured to acquire a blood pressure parameter of the user to be measured, acquire second target information of the user to be measured,
the identifying module 302, further configured to identify, according to the second target information of the user to be measured, a second blood pressure prediction affected condition corresponding to the second target information of the user to be measured, and identify, according to the second blood pressure prediction affected condition corresponding to the second target information of the user to be measured, a second blood pressure prediction affected level corresponding to the second target information of the user to be measured; and
a second judging module 308, configured to judge whether the second blood pressure prediction affected level is greater than or equal to a preset second affected level threshold;
a second correcting module 309, configured to correct the blood pressure parameter of the user to be measured according to the second target information if the judging result of the second judging module 308 is positive.

In this optional implementation, the second target information includes at least one of activity state, sleep state, and psychological state of the user to be measured, the activity state includes activity intensity and/or activity duration, and the sleep state includes sleep depth and/or sleep duration.

It is evident that, by implementing the apparatus of non-contact blood pressure measurement based on radar technology described in FIG. 5, it is identified the corresponding second blood pressure prediction affected condition according to the second target information of the user to be measured, and then judged whether it is necessary to further correct the blood pressure parameters of the user to be measured. In this case, the judgment comprehensiveness and accuracy of the blood pressure condition of the user to be measured can be improved, and then the blood pressure prediction deviation caused by the current state of the user to be measured and/or different self conditions can be effectively corrected, thereby improving the reliability and accuracy of blood pressure prediction. At the same time, in the network framework design, by rationally dividing the blood pressure prediction sub-model and the system correction sub-model, both the limitations of hardware storage and computing capacity are considered, and the optimal division of functions is achieved. This helps to reduce the cost and complexity of hardware deployment while maintaining the prediction accuracy.

In yet another optional implementation, the method of the detecting module performing a signal detection operation on a target body part of a user to be measured by means of a preset radar detector to acquire a signal parameter of an echo signal of the user to be measured specifically includes:
acquiring first detecting reference information of the user to be measured;
identifying, according to the first detecting reference information of the user to be measured, a detecting control parameter corresponding to the preset radar detector,
performing, according to the detecting control parameter corresponding to the radar detector, the signal detection operation on the target body part of the user to be measured to acquire the signal parameter of the echo signal of the user to be measured.

In this optional implementation, the first detecting reference information of the user to be measured includes health condition and/or current activity of the user to be measured, the current activity includes at least one of current activity intensity, current activity posture, current activity amplitude, and duration of activity already performed, and the detecting control parameter includes at least one of detecting position, detecting distance, detecting wave, detecting duration, and signal receiving position.

It is evident that, by implementing the apparatus of non-contact blood pressure measurement based on radar technology described in FIG. 5, it is identified the detecting control parameters corresponding to the radar detector based on the first detecting reference information of the user to be measured, and then perform signal detection on the target body part of the user to be measured, thus improving the control flexibility and accuracy of the radar detector, thereby improving the microwave signal transmission accuracy for the user to be measured and the echo signal reception accuracy for the user to be measured, thereby facilitating the subsequent accurate blood pressure prediction process.

In yet another optional implementation, the method of the detecting module 301 performing a signal detection operation on a target body part of a user to be measured by means of a preset radar detector to acquire a signal parameter of an echo signal of the user to be measured specifically includes:
before identifying a detecting control parameter corresponding to the preset radar detector according to the first detecting reference information of the user to be measured, acquiring second detecting reference information of the user to be measured;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured; and
judging whether the detecting affected level is greater than or equal to a preset detecting affected level threshold;
identifying a detecting control parameter corresponding to the preset radar detector according to the first detecting reference information and the second detecting reference information if a corresponding judging result is positive; and
triggering to perform identifying a detecting control parameter corresponding to the preset radar detector according to the first detecting reference information of the user to be measured if a corresponding judging result is negative.

In this optional embodiment, the second detecting reference information of the user to be measured includes clothing information and/or electronic device carrying information of the user to be measured, the clothing information includes at least one of clothing type, clothing thickness, clothing quantity, clothing wearing position, and clothing wearing tightness, and the electronic device carrying information includes at least one of electronic device type, electronic device location, and electronic device working state.

It is evident that, by implementing the apparatus of non-contact blood pressure measurement based on radar technology described in FIG. 5, it is identified further the detecting control parameters corresponding to the radar detector based on the second detecting reference information of the user to be measured, and then perform signal detection on the target body part of the user to be measured, thus further improving the control flexibility and accuracy of the radar detector, thereby is conducive to improving the microwave signal transmission accuracy for the user to be measured and the echo signal reception accuracy for the user to be measured, thereby facilitating the subsequent quick and accurate blood pressure prediction process.

In another optional implementation, the apparatus further includes:
a third acquiring module 310, configured to, before the identifying module 302 identifying biological signal information of the user to be measured according to the signal parameter of the echo signal, acquire a detecting feature parameter of the radar detector when performing the signal detection operation, and acquire an apparatus parameter of the radar detector;
the identifying module 302, further configured to identify, according to the detecting feature parameter and the apparatus parameter, a detection wave affected condition corresponding to the radar detector, and identify, according to the detection wave affected condition, a detection wave affected level corresponding to the radar detector;
a third judging module 311, configured to judge whether the detection wave affected level is greater than or equal to a preset detection wave affected level threshold; and,
the identifying module 302, further configured to identify, if a corresponding judging result of the third judging module 311 is positive, according to the detection wave affected condition, a signal processing parameter of the echo signal, and process, according to the signal processing parameter, the echo signal to update the echo signal.

In this optional implementation, the detecting feature parameter includes at least one of detecting jitter amplitude, detecting jitter frequency, and detecting jitter time, and the apparatus parameter of the radar detector includes apparatus model and/or apparatus loss of the radar detector.

It is evident that, by implementing the apparatus of non-contact blood pressure measurement based on radar technology described in FIG. 5, it is identified the corresponding detection wave affected condition according to the detection feature parameters and apparatus parameters of the radar detector, such as wave amplitude change and wave velocity change. Then, when the detection wave is greatly affected, the echo signal is processed according to the detection wave affected condition, which is conducive to improving the reliability and accuracy of the collected echo signal, thereby improving the analysis reliability and accuracy of the subsequent echo signal, and thus improving the efficiency and accuracy of the blood pressure measurement for the user to be measured.

### Fourth Embodiment

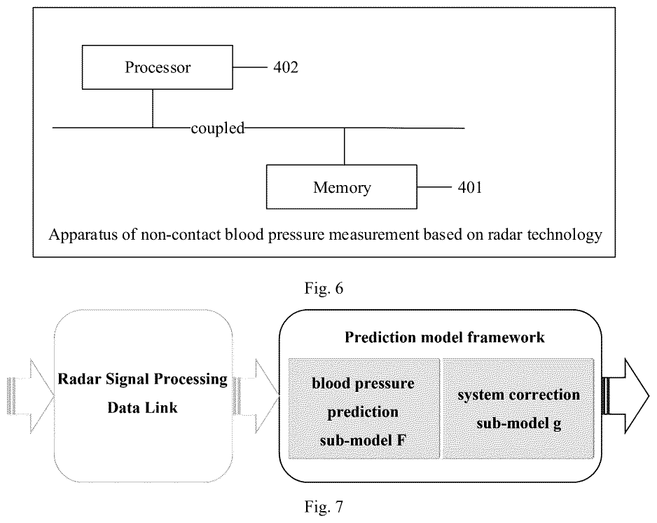

Referring to FIG. 6, FIG. 6 is a schematic structural diagram of another apparatus of non-contact blood pressure measurement based on radar technology disclosed in the present embodiment of the disclosure. As shown in FIG. 6, the apparatus of non-contact blood pressure measurement based on radar technology may include:
a memory 401, memorized with an executable code; and
a processor 402, coupled with the memory 401.

The processor 402 invokes the executable code memorized in the memory 401 to perform steps of the method of non-contact blood pressure measurement based on radar technology as described in first embodiment or second embodiment.

### Fifth Embodiment

Disclosed in the present embodiment of the disclosure is a non-transitory computer memory medium, the non-transitory computer memory medium memorizes computer instructions; when the computer instructions are invoked, steps of the method of non-contact blood pressure measurement based on radar technology described in the first embodiment and second embodiment of the present disclosure are performed.

### Sixth Embodiment

Disclosed in the present embodiment of the disclosure is a computer program product, the computer program product including a non-instantaneous computer readable memory medium memorized with a computer program. The computer program may be operated to enable the computer to perform steps in the method of non-contact blood pressure measurement based on radar technology described in the first embodiment or second embodiment.

The aforementioned described embodiment of the apparatus is only illustrative. The modules described as separate components may or may not be physically separated, and the modules used as components for display may or may not be physical modules, that is, they may be located in the same place or may be distributed to a plurality of network modules. Some or all these modules may be selected according to practical demands to achieve the purpose of the solution of the present embodiment. It may be understood and performed by a person of ordinary skill in the art without inventive effort.

With the specific description of the above embodiments, it is clear to those skilled in the art that the various implementations may be implemented with the aid of software plus the necessary common hardware platform, and admittedly, with the aid of hardware. Based on such an understanding, the above technical solutions that essentially or contribute to the prior art may be embodied in the form of a software product which may be memorized in a non-transitory computer-readable memory medium, the non-transitory memory medium including Read-Only Memory, Random Access Memory, Programmable Read-only Memory, Erasable Programmable Read Only Memory, One-time Programmable Read-Only Memory, Electrically-Erasable Programmable Read-Only Memory, Compact Disc Read-Only Memory, other Compact Disc Memory, Disk Memory, Tape Memory or any other non-transitory computer-readable medium that may be used to carry or memorize data.

Finally, it should be noted that the method and apparatus of non-contact blood pressure measurement based on radar technology disclosed in the embodiments of the present disclosure are only preferred embodiments of the present disclosure, and are only used to illustrate the technical solutions of the present disclosure, but not to limit them. Despite the detailed description of the disclosure with reference to the aforementioned embodiments, it should be understood, by those skilled in the art, that the technical solutions recorded in the aforementioned embodiments may still be modified, or equivalent substitutions for some of the technical features thereof may be made; which the essence of the corresponding technical solutions of these modifications or substitutions is without departing from the spirit and scope of the technical solutions of the various embodiments of the disclosure.

1. A method of non-contact blood pressure measurement based on radar technology, comprising:
performing a signal detection operation on a target body part of a user to be measured by means of a preset radar detector to acquire a signal parameter of an echo signal of the user to be measured;
identifying, according to the signal parameter of the echo signal, biological signal information of the user to be measured; and
performing, according to the biological signal information of the user to be measured, a blood pressure prediction operation on the user to be measured to acquire a blood pressure parameter of the user to be measured, wherein the blood pressure parameter comprises a systolic blood pressure parameter and/or a diastolic blood pressure parameter.

2. The method of non-contact blood pressure measurement based on radar technology according to claim 1, wherein the signal parameter of the echo signal comprises at least one of a waveform increasing rate, a waveform peak, a waveform decreasing rate, a waveform period, a waveform frequency, and a waveform amplitude of the echo signal; the biological signal information of the user to be measured comprises at least one of vascular elasticity degree, vascular blood flow rate, vascular blood flow resistance, vascular blood flow volume, and time of change of vascular blood flow of the user to be measured;
the performing, according to the biological signal information of the user to be measured, a blood pressure prediction operation on the user to be measured to acquire a blood pressure parameter of the user to be measured comprises:
identifying, according to the biological signal information of the user to be measured, cardiac feature information of the user to be measured; the cardiac feature information of the user to be measured comprising cardiac systolic feature information and/or cardiac diastolic feature information of the user to be measured; and
performing, according to the cardiac feature information of the user to be measured, a blood pressure prediction operation on the user to be measured to acquire a blood pressure parameter of the user to be measured.

3. The method of non-contact blood pressure measurement based on radar technology according to claim 1, wherein, after the performing, according to the biological signal information of the user to be measured, a blood pressure prediction operation on the user to be measured to acquire a blood pressure parameter of the user to be measured, the method further comprises:
acquiring first target information of the user to be measured, the first target information comprising basic information and/or health condition information of the user to be measured, the basic information of the user to be measured comprising at least one of height, weight, gender, and body type of the user to be measured;
identifying, according to the first target information of the user to be measured, a first blood pressure prediction affected condition corresponding to the first target information of the user to be measured, and identifying, according to the first blood pressure prediction affected condition corresponding to the first target information of the user to be measured, a first blood pressure prediction affected level corresponding to the first target information of the user to be measured; and
judging whether the first blood pressure prediction affected level is greater than or equal to a preset first affected level threshold, and correcting the blood pressure parameter of the user to be measured according to the first target information if yes.

4. The method of non-contact blood pressure measurement based on radar technology according to claim 1, wherein after the performing, according to the biological signal information of the user to be measured, a blood pressure prediction operation on the user to be measured to acquire a blood pressure parameter of the user to be measured, the method further comprises:
acquiring second target information of the user to be measured, the second target information comprising at least one of activity state, sleep state, and psychological state of the user to be measured, the activity state comprising activity intensity and/or activity duration, and the sleep state comprising sleep depth and/or sleep duration;
identifying, according to the second target information of the user to be measured, a second blood pressure prediction affected condition corresponding to the second target information of the user to be measured, and identifying, according to the second blood pressure prediction affected condition corresponding to the second target information of the user to be measured, a second blood pressure prediction affected level corresponding to the second target information of the user to be measured; and
judging whether the second blood pressure prediction affected level is greater than or equal to a preset second affected level threshold, and if yes, correcting the blood pressure parameter of the user to be measured according to the second target information.

5. The method of non-contact blood pressure measurement based on radar technology according to claim 1, wherein the performing a signal detection operation on a target body part of a user to be measured by means of a preset radar detector to acquire a signal parameter of an echo signal of the user to be measured comprises:
acquiring first detecting reference information of the user to be measured, the first detecting reference information of the user to be measured comprising health condition and/or current activity of the user to be measured, the current activity comprising at least one of current activity intensity, current activity posture, current activity amplitude, and duration of activity already performed;
identifying a detecting control parameter corresponding to the preset radar detector according to the first detecting reference information of the user to be measured, the detecting control parameter comprising at least one of detecting position, detecting distance, detecting wave, detecting duration, and signal receiving position; and
performing, according to the detecting control parameter corresponding to the radar detector, the signal detection operation on the target body part of the user to be measured to acquire the signal parameter of the echo signal of the user to be measured.

6. The method of non-contact blood pressure measurement based on radar technology according to claim 2, wherein the performing a signal detection operation on a target body part of a user to be measured by means of a preset radar detector to acquire a signal parameter of an echo signal of the user to be measured comprises:
acquiring first detecting reference information of the user to be measured, the first detecting reference information of the user to be measured comprising health condition and/or current activity of the user to be measured, the current activity comprising at least one of current activity intensity, current activity posture, current activity amplitude, and duration of activity already performed;
identifying a detecting control parameter corresponding to the preset radar detector according to the first detecting reference information of the user to be measured, the detecting control parameter comprising at least one of detecting position, detecting distance, detecting wave, detecting duration, and signal receiving position; and
performing, according to the detecting control parameter corresponding to the radar detector, the signal detection operation on the target body part of the user to be measured to acquire the signal parameter of the echo signal of the user to be measured.

7. The method of non-contact blood pressure measurement based on radar technology according to claim 3, wherein the performing a signal detection operation on a target body part of a user to be measured by means of a preset radar detector to acquire a signal parameter of an echo signal of the user to be measured comprises:
acquiring first detecting reference information of the user to be measured, the first detecting reference information of the user to be measured comprising health condition and/or current activity of the user to be measured, the current activity comprising at least one of current activity intensity, current activity posture, current activity amplitude, and duration of activity already performed;
identifying a detecting control parameter corresponding to the preset radar detector according to the first detecting reference information of the user to be measured, the detecting control parameter comprising at least one of detecting position, detecting distance, detecting wave, detecting duration, and signal receiving position; and
performing, according to the detecting control parameter corresponding to the radar detector, the signal detection operation on the target body part of the user to be measured to acquire the signal parameter of the echo signal of the user to be measured.

8. The method of non-contact blood pressure measurement based on radar technology according to claim 4, wherein the performing a signal detection operation on a target body part of a user to be measured by means of a preset radar detector to acquire a signal parameter of an echo signal of the user to be measured comprises:
acquiring first detecting reference information of the user to be measured, the first detecting reference information of the user to be measured comprising health condition and/or current activity of the user to be measured, the current activity comprising at least one of current activity intensity, current activity posture, current activity amplitude, and duration of activity already performed;
identifying a detecting control parameter corresponding to the preset radar detector according to the first detecting reference information of the user to be measured, the detecting control parameter comprising at least one of detecting position, detecting distance, detecting wave, detecting duration, and signal receiving position; and
performing, according to the detecting control parameter corresponding to the radar detector, the signal detection operation on the target body part of the user to be measured to acquire the signal parameter of the echo signal of the user to be measured.

9. The method of non-contact blood pressure measurement based on radar technology according to claim 5, wherein, before identifying a detecting control parameter corresponding to the preset radar detector according to the first detecting reference information of the user to be measured, the method further comprises:
acquiring second detecting reference information of the user to be measured, the second detecting reference information of the user to be measured comprising clothing information and/or electronic device carrying information of the user to be measured, the clothing information comprising at least one of clothing type, clothing thickness, clothing quantity, clothing wearing position, and clothing wearing tightness, and the electronic device carrying information comprising at least one of electronic device type, electronic device location, and electronic device working state;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured;
judging whether the detecting affected level is greater than or equal to a preset detecting affected level threshold;
identifying a detecting control parameter corresponding to the preset radar detector according to the first detecting reference information and the second detecting reference information if a corresponding judging result is positive; and
triggering to perform identifying a detecting control parameter corresponding to the preset radar detector according to the first detecting reference information of the user to be measured if a corresponding judging result is negative.

10. The method of non-contact blood pressure measurement based on radar technology according to claim 6, wherein, before identifying a detecting control parameter corresponding to the preset radar detector according to the first detecting reference information of the user to be measured, the method further comprises:
acquiring second detecting reference information of the user to be measured, the second detecting reference information of the user to be measured comprising clothing information and/or electronic device carrying information of the user to be measured, the clothing information comprising at least one of clothing type, clothing thickness, clothing quantity, clothing wearing position, and clothing wearing tightness, and the electronic device carrying information comprising at least one of electronic device type, electronic device location, and electronic device working state;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured;
judging whether the detecting affected level is greater than or equal to a preset detecting affected level threshold;
identifying a detecting control parameter corresponding to the preset radar detector according to the first detecting reference information and the second detecting reference information if a corresponding judging result is positive; and
triggering to perform identifying a detecting control parameter corresponding to the preset radar detector according to the first detecting reference information of the user to be measured if a corresponding judging result is negative.

11. The method of non-contact blood pressure measurement based on radar technology according to claim 7, wherein, before identifying a detecting control parameter corresponding to the preset radar detector according to the first detecting reference information of the user to be measured, the method further comprises:
acquiring second detecting reference information of the user to be measured, the second detecting reference information of the user to be measured comprising clothing information and/or electronic device carrying information of the user to be measured, the clothing information comprising at least one of clothing type, clothing thickness, clothing quantity, clothing wearing position, and clothing wearing tightness, and the electronic device carrying information comprising at least one of electronic device type, electronic device location, and electronic device working state;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured;
judging whether the detecting affected level is greater than or equal to a preset detecting affected level threshold;
identifying a detecting control parameter corresponding to the preset radar detector according to the first detecting reference information and the second detecting reference information if a corresponding judging result is positive; and
triggering to perform identifying a detecting control parameter corresponding to the preset radar detector according to the first detecting reference information of the user to be measured if a corresponding judging result is negative.

12. The method of non-contact blood pressure measurement based on radar technology according to claim 8, wherein, before identifying a detecting control parameter corresponding to the preset radar detector according to the first detecting reference information of the user to be measured, the method further comprises:
acquiring second detecting reference information of the user to be measured, the second detecting reference information of the user to be measured comprising clothing information and/or electronic device carrying information of the user to be measured, the clothing information comprising at least one of clothing type, clothing thickness, clothing quantity, clothing wearing position, and clothing wearing tightness, and the electronic device carrying information comprising at least one of electronic device type, electronic device location, and electronic device working state;
identifying, according to the second detecting reference information of the user to be measured, a detecting affected condition corresponding to the second detecting reference information of the user to be measured, and identifying, according to the detecting affected condition corresponding to the second detecting reference information of the user to be measured, a detecting affected level corresponding to the second detecting reference information of the user to be measured;
judging whether the detecting affected level is greater than or equal to a preset detecting affected level threshold;
identifying a detecting control parameter corresponding to the preset radar detector according to the first detecting reference information and the second detecting reference information if a corresponding judging result is positive; and
triggering to perform identifying a detecting control parameter corresponding to the preset radar detector according to the first detecting reference information of the user to be measured if a corresponding judging result is negative.

13. The method of non-contact blood pressure measurement based on radar technology according to claim 9, wherein, before the identifying, according to the signal parameter of the echo signal, biological signal information of the user to be measured, the method further comprises:
acquiring a detecting feature parameter of the radar detector when performing the signal detection operation, the detecting feature parameter comprising at least one of detecting jitter amplitude, detecting jitter frequency, and detecting jitter time;
acquiring an apparatus parameter of the radar detector and identifying, according to the detecting feature parameter and the apparatus parameter, a detection wave affected condition corresponding to the radar detector, the apparatus parameter of the radar detector comprising apparatus model and/or apparatus loss of the radar detector;
identifying, according to the detection wave affected condition, a detection wave affected level corresponding to the radar detector, and judging whether the detection wave affected level is greater than or equal to a preset detection wave affected level threshold; and,
if a corresponding judging result is positive, identifying, according to the detection wave affected condition, a signal processing parameter of the echo signal, and processing, according to the signal processing parameter, the echo signal to update the echo signal.

14. The method of non-contact blood pressure measurement based on radar technology according to claim 10, wherein, before the identifying, according to the signal parameter of the echo signal, biological signal information of the user to be measured, the method further comprises:
acquiring a detecting feature parameter of the radar detector when performing the signal detection operation, the detecting feature parameter comprising at least one of detecting jitter amplitude, detecting jitter frequency, and detecting jitter time;
acquiring an apparatus parameter of the radar detector and identifying, according to the detecting feature parameter and the apparatus parameter, a detection wave affected condition corresponding to the radar detector, the apparatus parameter of the radar detector comprising apparatus model and/or apparatus loss of the radar detector;
identifying, according to the detection wave affected condition, a detection wave affected level corresponding to the radar detector, and judging whether the detection wave affected level is greater than or equal to a preset detection wave affected level threshold; and,
if a corresponding judging result is positive, identifying, according to the detection wave affected condition, a signal processing parameter of the echo signal, and processing, according to the signal processing parameter, the echo signal to update the echo signal.

15. The method of non-contact blood pressure measurement based on radar technology according to claim 11, wherein, before the identifying, according to the signal parameter of the echo signal, biological signal information of the user to be measured, the method further comprises:
acquiring a detecting feature parameter of the radar detector when performing the signal detection operation, the detecting feature parameter comprising at least one of detecting jitter amplitude, detecting jitter frequency, and detecting jitter time;
acquiring an apparatus parameter of the radar detector and identifying, according to the detecting feature parameter and the apparatus parameter, a detection wave affected condition corresponding to the radar detector, the apparatus parameter of the radar detector comprising apparatus model and/or apparatus loss of the radar detector;
identifying, according to the detection wave affected condition, a detection wave affected level corresponding to the radar detector, and judging whether the detection wave affected level is greater than or equal to a preset detection wave affected level threshold; and,
if a corresponding judging result is positive, identifying, according to the detection wave affected condition, a signal processing parameter of the echo signal, and processing, according to the signal processing parameter, the echo signal to update the echo signal.

16. The method of non-contact blood pressure measurement based on radar technology according to claim 12, wherein, before the identifying, according to the signal parameter of the echo signal, biological signal information of the user to be measured, the method further comprises:
acquiring a detecting feature parameter of the radar detector when performing the signal detection operation, the detecting feature parameter comprising at least one of detecting jitter amplitude, detecting jitter frequency, and detecting jitter time;
acquiring an apparatus parameter of the radar detector and identifying, according to the detecting feature parameter and the apparatus parameter, a detection wave affected condition corresponding to the radar detector, the apparatus parameter of the radar detector comprising apparatus model and/or apparatus loss of the radar detector;
identifying, according to the detection wave affected condition, a detection wave affected level corresponding to the radar detector, and judging whether the detection wave affected level is greater than or equal to a preset detection wave affected level threshold; and,
if a corresponding judging result is positive, identifying, according to the detection wave affected condition, a signal processing parameter of the echo signal, and processing, according to the signal processing parameter, the echo signal to update the echo signal.

17. An apparatus of non-contact blood pressure measurement based on radar technology, wherein the apparatus comprises:
a memory, memorized with an executable code; and
a processor, coupled with the memory,
wherein the processor invokes the executable code memorized in the memory to perform a method of non-contact blood pressure measurement based on radar technology,
the method of non-contact blood pressure measurement based on radar technology comprising:
performing a signal detection operation on a target body part of a user to be measured by means of a preset radar detector to acquire a signal parameter of an echo signal of the user to be measured;
identifying, according to the signal parameter of the echo signal, biological signal information of the user to be measured; and
performing, according to the biological signal information of the user to be measured, a blood pressure prediction operation on the user to be measured to acquire a blood pressure parameter of the user to be measured, wherein the blood pressure parameter comprises a systolic blood pressure parameter and/or a diastolic blood pressure parameter.

18. The apparatus of non-contact blood pressure measurement based on radar technology according to claim 17, wherein the signal parameter of the echo signal comprises at least one of a waveform increasing rate, a waveform peak, a waveform decreasing rate, a waveform period, a waveform frequency, and a waveform amplitude of the echo signal; the biological signal information of the user to be measured comprises at least one of vascular elasticity degree, vascular blood flow rate, vascular blood flow resistance, vascular blood flow volume, and time of change of vascular blood flow of the user to be measured;
the performing, according to the biological signal information of the user to be measured, a blood pressure prediction operation on the user to be measured to acquire a blood pressure parameter of the user to be measured comprises:
identifying, according to the biological signal information of the user to be measured, cardiac feature information of the user to be measured; the cardiac feature information of the user to be measured comprising cardiac systolic feature information and/or cardiac diastolic feature information of the user to be measured; and
performing, according to the cardiac feature information of the user to be measured, a blood pressure prediction operation on the user to be measured to acquire a blood pressure parameter of the user to be measured.

19. A non-transitory computer memory medium, wherein the non-transitory computer memory medium memorizes computer instructions; when the computer instructions are invoked, a method of non-contact blood pressure measurement based on radar technology is performed,
the method of non-contact blood pressure measurement based on radar technology comprising:
performing a signal detection operation on a target body part of a user to be measured by means of a preset radar detector to acquire a signal parameter of an echo signal of the user to be measured;
identifying, according to the signal parameter of the echo signal, biological signal information of the user to be measured; and
performing, according to the biological signal information of the user to be measured, a blood pressure prediction operation on the user to be measured to acquire a blood pressure parameter of the user to be measured, wherein the blood pressure parameter comprises a systolic blood pressure parameter and/or a diastolic blood pressure parameter.

20. The non-transitory computer memory medium according to claim 19, wherein the signal parameter of the echo signal comprises at least one of a waveform increasing rate, a waveform peak, a waveform decreasing rate, a waveform period, a waveform frequency, and a waveform amplitude of the echo signal; the biological signal information of the user to be measured comprises at least one of vascular elasticity degree, vascular blood flow rate, vascular blood flow resistance, vascular blood flow volume, and time of change of vascular blood flow of the user to be measured;
the performing, according to the biological signal information of the user to be measured, a blood pressure prediction operation on the user to be measured to acquire a blood pressure parameter of the user to be measured comprises:
identifying, according to the biological signal information of the user to be measured, cardiac feature information of the user to be measured; the cardiac feature information of the user to be measured comprising cardiac systolic feature information and/or cardiac diastolic feature information of the user to be measured; and
performing, according to the cardiac feature information of the user to be measured, a blood pressure prediction operation on the user to be measured to acquire a blood pressure parameter of the user to be measured.
